SEQUENCE LISTING (3 sequences)

SEQ ID NO: 1 - DNA, 15 bp, artificial
  probe
tgttctcttgtcttg

SEQ ID NO: 2 - DNA, 15 bp, artificial
  probe
caagacaagagaaca

SEQ ID NO: 3 - DNA, 15 bp, artificial
  probe
ttttttttttttttt

(19) United States
(12) Patent Application Publication
(10) Pub. No.: US 2007/0111224 A1
(43) Pub. Date: May 17, 2007

(54) Electrochemical sensor for detecting biomolecule, metthod of manufacturing the same, and method of detecting biomolecule using the same

(76) Inventors: Sung-ouk Jung, Suwon-si (KR); Soo-suk Lee, Suwon-si (KR)

Correspondence Address: CANTOR COLBURN, LLP, 55 GRIFFIN ROAD SOUTH, BLOOMFIELD, CT, 06002, US

(21) Appl. No.: 11/449,976

(22) Filed: Jun. 9, 2006

(30) Foreign Application Priority Data
May 12, 2005 (KR) 10-2005-0117688

(51) Int. Cl. C12Q 1/68; C12M 3/00

(52) U.S. Cl. 435/6; 435/287.2; 977/924

(57) ABSTRACT

Provided is a sensor for detecting a biomolecule, which includes: a substrate; a first material, immobilized on the substrate, having a cavity structure; a secondary material capable of selectively binding to the cavity structure of the first material and having electrochemical activity; and a probe biomolecule immobilized to the secondary material. A method of manufacturing the biomolecule detection sensor and a method of detecting a biomolecule using the biomolecule detection sensor are also provided. Therefore, a target biomolecule can be easily detected using an electrochemical reaction with high accuracy within a short time. Furthermore, there is no need to elaborately design probes, and thus, the biomolecule detection sensor can be easily manufactured. In addition, label-free detection is possible, which simplifies a detection process, and there is no need to use expensive auxiliary equipment.

### CROSS-REFERENCE TO RELATED PATENT APPLICATION

This application claims priority from Korean Patent Application No. 10-2005-0117688, filed on Dec. 5, 2005, in the Korean Intellectual Property Office, the disclosure of which is incorporated herein in its entirety by reference.

### FIELD OF THE INVENTION

The present invention relates to an electrochemical sensor for detecting a biomolecule, and more particularly, to an electrochemical biosensor for label-free and prompt detection of a biomolecule, a method of manufacturing the same, and a method of detecting a biomolecule using the same.

### DESCRIPTION OF THE RELATED ART

Effective methods for detection of biomolecules are required in various fields. The biochip technology field is a representative field for biomolecule detection. Biochips are tools where a high-density array of probe biomolecules, such as DNAs or proteins, is attached onto a substrate, and can analyze gene expression profile, gene defects, protein distribution, and reaction profile in samples. The biochips can be divided into microarray chips where probe biomolecules are attached to a solid substrate and lab-on-a-chips where probe biomolecules are attached to a microchannel. The biochips require a system capable of detecting binding events of target biomolecules with probe biomolecules immobilized on a substrate to thereby identify the presence of target biomolecules capable of binding with probe biomolecules in a sample.

Generally, the reading of a DNA chip for gene assay is based on fluorescence detection which includes: labeling sample DNAs with fluorescent dyes, allowing the sample DNAs to react with probes on the chip, and detecting fluorescently marked regions on a surface of the chip using a confocal microscope or a Charge Coupled Device (CCD) camera (see U.S. Pat. No. 6,141,096). However, the fluorescence detection method prohibits its use on a small-sized chip, and cannot provide a digitized output.

Biomolecule binding events, such as DNA hybridization, can also be detected using an electrochemical detection method.

For example, there is a method of detecting a target nucleic acid using a DNA wrap assay, instead of a conventional sandwich assay (Chad E. Immoos, Stephen J. Lee, and Mark W. Grinstaff, J. AM. CHEM. SOC., 126: 10814-10815, 2004). According to this method, capture strands are immobilized on a substrate, and a linker, probe strands, and an electrochemically active material are sequentially linked to the capture strands. When target strands bind to the probe strands, the electrochemically active material approaches the substrate, thereby changing a cyclic voltammogram.

There is also a method of detecting a target nucleic acid using probe strands with a hairpin structure (Chunhai Fan, Kevin W. Plaxco, and Alan J. Heeger, PNAS, 100: 9134-9137, 2003). According to this method, hairpin probe strands with an electrochemically active material are immobilized on a substrate. When target strands bind to the probe strands, the hairpin structure is disrupted and the electrochemically active material becomes away from the substrate, thereby changing a cyclic voltammogram.

There is also a method of detecting a target nucleic acid using the competitive hybridization between DNAs (see U.S. Patent Publication No. 20040110214). According to this method, probe DNAs are immobilized on a substrate, and signaling DNAs with an electrochemically active material are hybridized to the probe DNAs. Target DNAs are competitively hybridized to the probe DNAs and the signaling DNAs. As a result, the signaling DNAs are released from the substrate, thereby changing a cyclic voltammogram.

However, the above-described electrochemical detection methods for target biomolecules require a very precise design and construction of probe biomolecules or signaling biomolecules, and take an extended detection time.

### SUMMARY OF THE INVENTION

The present invention has been made in view of the above problems.

Therefore, the present invention provides a sensor which is easily designed for label-free and prompt detection of a biomolecule.

The present invention also provides a method of manufacturing the sensor.

The present invention also provides a method of detecting a biomolecule using the sensor.

According to an aspect of the present invention, there is provided a sensor for detecting a biomolecule, the sensor including: a substrate; a first material, immobilized on the substrate, having a cavity structure; a secondary material capable of selectively binding to the cavity structure of the first material and having electrochemical activity; and a probe biomolecule immobilized to the secondary material.

The substrate may be selected from the group consisting of silicone wafer, glass, quartz, metal, and plastic.

Gold may be coated on a surface of the substrate.

The first material may be cyclodextrin or calixarene, and the secondary material may be metallocene (e.g., ferrocene), alkylammonium, or a compound containing an adamantly group.

The first material may be immobilized on the substrate using a self-assembly process.

The biomolecule may be a nucleic acid or a protein.

The nucleic acid may be selected from the group consisting of DNA, RNA, Peptide Nucleic Acid (PNA), Locked Nucleic Acid (LNA), and a hybrid thereof.

According to another aspect of the present invention, there is provided a method of manufacturing a biomolecule detection sensor, the method including: immobilizing a first material having a cavity structure on a substrate; immobilizing a probe biomolecule to a secondary material capable of selectively binding to the cavity structure of the first material and having electrochemical activity; and providing the secondary material to the first material so that the secondary material selectively binds to the first material.

The method may further include providing a capping material to the substrate so that a first material-free surface of the substrate is capped by the capping material, after the immobilization of the first material on the substrate.

The first material may be β-cyclodextrin and the secondary material may be ferrocene.

The immobilization of the first material on the substrate may be performed using a self-assembly process.

According to still another aspect of the present invention, there is provided a method of detecting a biomolecule, the method including: providing a sample suspected of containing a target biomolecule to a biomolecule detection sensor including a substrate; a first material, immobilized on the substrate, having a cavity structure; a secondary material capable of selectively binding to the cavity structure of the first material and having electrochemical activity; and a probe biomolecule immobilized to the secondary material; and measuring a change in current before and after providing the sample.

The measuring of the change in current may be performed using cyclic voltammetry.

When the target biomolecule is present in the sample, the target biomolecule may bind to the probe biomolecule, the secondary material may be separated from the first material, and a current may be changed.

According to the present invention, a target biomolecule can be easily detected using an electrochemical reaction with high accuracy within a short time. Furthermore, there is no need to elaborately design probes, and thus, it is easy to manufacture a detection sensor. In addition, label-free detection is possible, which simplifies a detection process, and there is no need to use expensive auxiliary equipment.

### BRIEF DESCRIPTION OF THE DRAWINGS

The above and other features and advantages of the present invention will become more apparent by describing in detail exemplary embodiments thereof with reference to the attached drawings in which:

FIG. 1 schematically illustrates the structure of a biomolecule detection sensor according to an embodiment of the present invention;

FIG. 2 schematically illustrates a method of manufacturing a biomolecule detection sensor according to an embodiment of the present invention;

FIG. 3A is a graph illustrating a change in surface reflectance during immobilization of a first material and surface capping of a substrate in a method of manufacturing a biomolecule detection sensor according to an embodiment of the present invention;

FIG. 3B is a graph illustrating a change in surface reflectance with respect to time and incident angle during selective binding of a first material with a secondary material in a method of manufacturing a biomolecule detection sensor according to an embodiment of the present invention;

FIG. 3C is a graph illustrating a change in surface reflectance with time during selective binding of a first material with a secondary material in a method of manufacturing a biomolecule detection sensor according to an embodiment of the present invention;

FIG. 4 is a diagram illustrating a method of detecting a biomolecule using a biomolecule detection sensor according to an embodiment of the present invention;

FIG. 5 is a graph illustrating redox peaks of a hybridization buffer according to an example of the present invention and a conventional electrolyte solution.

FIG. 6 is a cyclic voltammogram with time when a hybridization buffer containing no DNA is provided to a biomolecule detection sensor according to an embodiment of the present invention;

FIG. 7 is a cyclic voltammogram with time when a hybridization buffer containing a target DNA perfectly matching with a probe DNA is provided to a biomolecule detection sensor according to an embodiment of the present invention;

FIG. 8 is a cyclic voltammogram with time when a hybridization buffer containing a DNA perfectly mismatching with a probe DNA is provided to a biomolecule detection sensor according to an embodiment of the present invention;

FIG. 9 illustrates a comparison of the cyclic voltammograms of FIGS. 6 through 8 (Fc-DNA: FIG. 6, comp-DNA: FIG. 7, mis-DNA: FIG. 8).

FIG. 10 is a graph illustrating a change in current with time for the cyclic votammograms of FIGS. 6 through 8 (Fc-DNA: FIG. 6, comp-DNA: FIG. 7, mis-DNA: FIG. 8);

FIG. 11 is a graph illustrating a current shift for 2 minutes for the cyclic votammograms of FIGS. 6 through 8 (Fc-DNA: FIG. 6, comp-DNA: FIG. 7, mis-DNA: FIG. 8); and

FIG. 12 is a graph illustrating a current measured every 10 seconds for 2 minutes for the cyclic votammograms of FIGS. 6 through 8 (Fc-DNA: FIG. 6, comp-DNA: FIG. 7, mis-DNA: FIG. 8).

### DETAILED DESCRIPTION OF THE INVENTION

The present invention will now be described more fully with reference to the accompanying drawings, in which exemplary embodiments of the invention are shown.

The present invention provides a biomolecule detection sensor using a first material having a cavity structure and a material capable of selectively binding to the cavity structure.

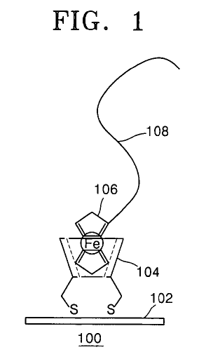

FIG. 1 schematically illustrates the structure of a biomolecule detection sensor according to an embodiment of the present invention.

Referring to FIG. 1, a biomolecule detection sensor 100 includes a substrate 102; a first material 104 which is immobilized on the substrate 102 and has a cavity structure; a secondary material 106 which can selectively bind to the cavity structure of the first material 104 and has electrochemical activity; and a probe biomolecule 108 which is immobilized to the secondary material 106.

There is no particular limitation to the type of the substrate 102. For example, the substrate 102, when used together with a separate operating electrode (not shown), may be an insulating substrate. Thus, the substrate 102 may be selected from the group consisting of silicon wafer, glass, quartz, and plastic.

On the other hand, the substrate 102, when used as an operating electrode, may be a conductive substrate. Thus, the substrate 102 may be made of metal, in particular gold, or may be a gold-coated substrate.

The first material 104 having the cavity structure can be immobilized on the substrate 102 using one of conventional methods. For example, the first material 104 can be immobilized on the substrate 102 via a self-assembly process by introducing a thiol group, an amine group, a silane group, or biotin, preferably a thiol group, at an end of the first material 104.

The secondary material 106 can selectively bind to the cavity structure of the first material 104 and has electrochemical activity. The secondary material 106 induces an electrochemical reaction to provide a cyclic voltammogram of the electrochemical reaction to thereby detect the presence or concentration of a target biomolecule.

In the present invention, the first material 104 and the secondary material 106 are not particularly limited provided that the first material 104 has a cavity structure, and the secondary material 106 can selectively bind to the cavity structure and has electrochemical activity.

For example, the first material 104 may be cyclodextrin or calixarene, and the secondary material 106 may be metallocene (e.g., ferrocene), alkylammonium, or a compound containing an adamantly group. In particular, the first material 104 and the secondary material 106 may be β-cyclodextrin and ferrocene, respectively.

FIG. 1 illustrates that β-cyclodextrin and ferrocene are respectively used as the first material 104 and the secondary material 106.

β-cyclodextrin (hereinafter, also called “CD”) has a crown or cavity structure and is represented by Formula I below:

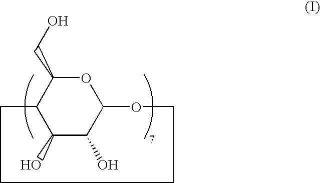

Ferrocene (hereinafter, also called “Fc”) is a representative compound of metallocene which is an aromatic transition metal complex, and an iron complex with two coordinated cyclopentadiene ligands. Ferrocene induces an electrochemical reaction to provide a cyclic voltammogram of the electrochemical reaction, which makes it possible to detect the presence or concentration of a target biomolecule.

The probe biomolecule 108 may be immobilized to the secondary material 106 via a linker. The linker is not particularly limited. For example, the linker may be —CO₂NH(CH₂)₆—.

The probe biomolecule 108 is designed to hybridize with a target biomolecule.

In the present invention, a biomolecule may be a nucleic acid or a protein. The “nucleic acid” is meant to comprehend various nucleic acids, nucleic acid analogues, and hybrids thereof. For example, the nucleic acid may be selected from the group consisting of DNA, RNA, Peptide Nucleic Acid (PNA), Locked Nucleic Acid (LNA), and a hybrid thereof. The nucleic acid may also be an oligonucleotide or a Polymerase Chain Reaction (PCR) product.

The present invention also provides a method of manufacturing the above-described biomolecule detection sensor.

The method of manufacturing the biomolecule detection sensor includes immobilizing a first material having a cavity structure on a substrate; immobilizing a probe biomolecule to a secondary material capable of selectively binding to the cavity structure of the first material and having electrochemical activity; and providing the secondary material to the first material so that the secondary material selectively binds to the first material.

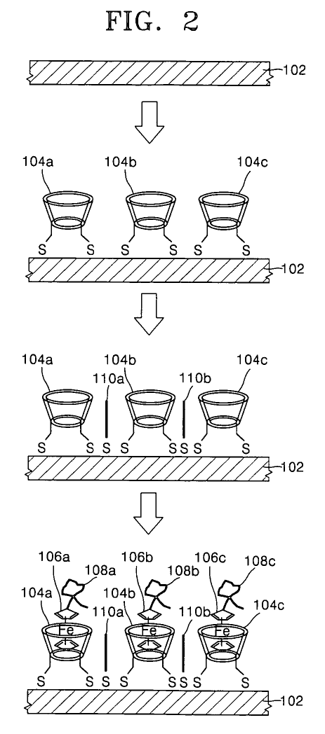

FIG. 2 schematically illustrates sequential processes for manufacturing a biomolecule detection sensor according to an embodiment of the present invention.

Referring to FIG. 2, first, first materials 104a, 104b, and 104c having cavity structures are immobilized on a substrate 102. FIG. 2 illustrates that β-cyclodextrin is used as the first materials 104a, 104b, and 104c.

The first materials 104a, 104b, and 104c may be immobilized on the substrate 102 by a self-assembly process. For example, a thiol group, an amine group, a silane group, or biotin, preferably a thiol group, may be introduced at ends of the first materials 104a, 104b, and 104c, and then, the first materials 104a, 104b, and 104c may be self-assembled onto the substrate 102. FIG. 2 illustrates that thiol-modified β-cyclodextrin is immobilized on the substrate 102 to form a self-assembled monolayer.

Next, capping materials 110a and 110b may be optionally provided on the substrate 102 so that a first material-free surface of the substrate 102 is capped. By doing so, a side reaction between the substrate 102 and a foreign substance that may be caused during detection of a target biomolecule can be prevented.

The capping materials 110a and 110b are not particularly limited. For example, a thiol-modified straight alkane chain C₁₂ (C₁₂—SH) where an end of a straight alkane chain C₁₂ is substituted by a thiol group may be used as the capping materials 110a and 110b.

Next, probe biomolecules 108a, 108b, and 108c are immobilized to secondary materials 106a, 106b, and 106c capable of selectively binding to the cavities of the first materials 104a, 104b, and 104c and having electrochemical activity, which is not illustrated in FIG. 2.

The probe biomolecules 108a, 108b, and 108c may be immobilized to the secondary materials 106a, 106b, and 106c via a linker. The linker is not particularly limited. For example, the linker may be —CO₂NH(CH₂)₆—.

Next, the secondary materials 106a, 106b, and 106c are provided to the first materials 104a, 104b, and 104c to selectively bind the secondary materials 106a, 106b, and 106c to the first materials 104a, 104b, and 104c. This completes a biomolecule detection sensor according to the present invention. FIG. 2 illustrates that ferrocene capable of selectively binding to β-cyclodextrin used as the first materials 104a, 104b, and 104c is used as the secondary materials 106a, 106b, and 106c.

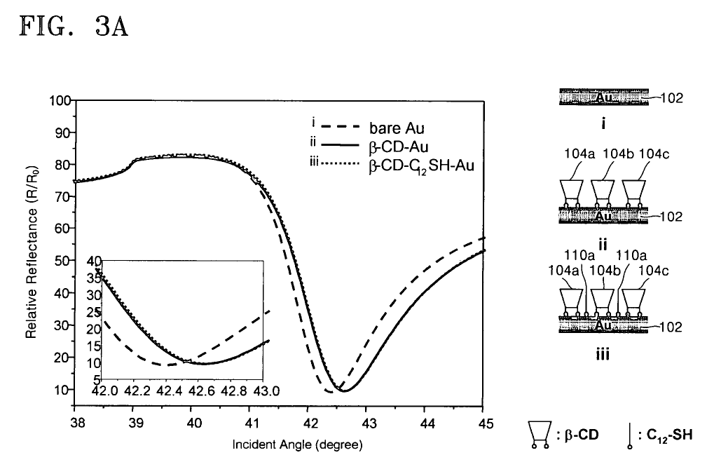

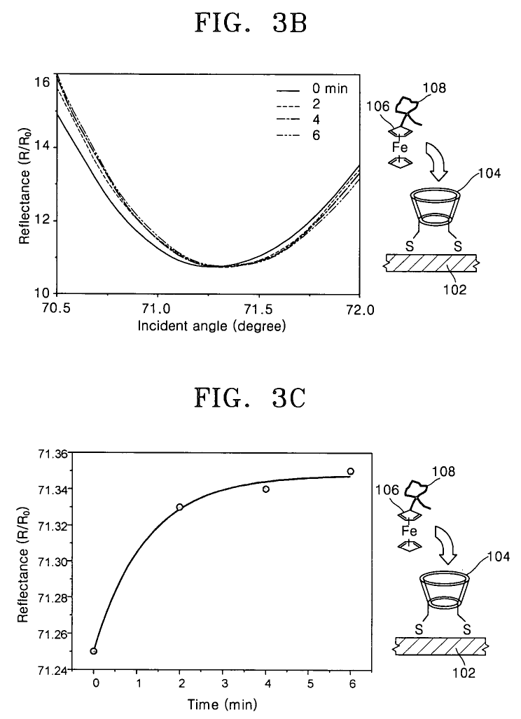

In an experimental example according to the present invention, a surface reflectance was measured during immobilization of a first material on a substrate, surface capping of the substrate, and selective binding of the first material with a secondary material, and the results are shown in FIGS. 3A, 3B, and 3C. Referring to FIG. 3A, there was a significant difference in surface reflectance before and after the immobilization of the first material on the substrate, whereas there was no significant difference in surface reflectance before and after the surface capping. Referring to FIGS. 3B and 3C, the selective binding of the first material with the secondary material was saturated about 6 minutes after providing the secondary material.

The present invention also provides a method of detecting a target biomolecule using the above-described biomolecule detection sensor.

The biomolecule detection method includes providing a sample suspected of containing a target biomolecule to a biomolecule detection sensor of the present invention; and measuring a change in current before and after providing the sample.

In detail, in the provision of the sample suspected of containing the target biomolecule to the biomolecule detection sensor of the present invention, a conventional hybridization buffer may be used. For example, the hybridization buffer may be a 1× SSPET buffer.

The measuring of the change in current before and after providing the sample may be performed using cyclic voltammetry. For example, there may be used a triode electrode system where a substrate of the biomolecule detection sensor of the present invention is used as an operating electrode, Ag/AgCl as a reference electrode, and a Pt wire as an auxiliary electrode. When an appropriate voltage capable of acting on a secondary material of the biomolecule detection sensor is applied to the operating electrode using a potentiostat, a cyclic voltammogram can be obtained.

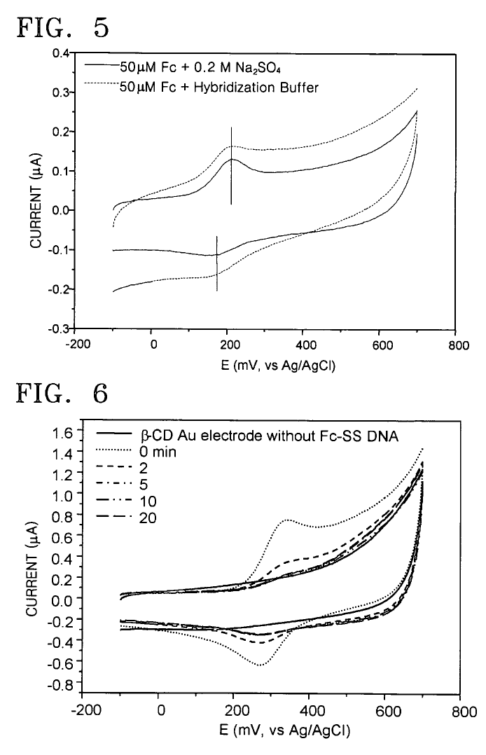

In the biomolecule detection method of the present invention, the cyclic voltammogram can be obtained using the hybridization buffer, without using a separate electrolyte solution (see FIG. 5).

If a significant change in current is observed, it can be determined that a target biomolecule is present in a sample.

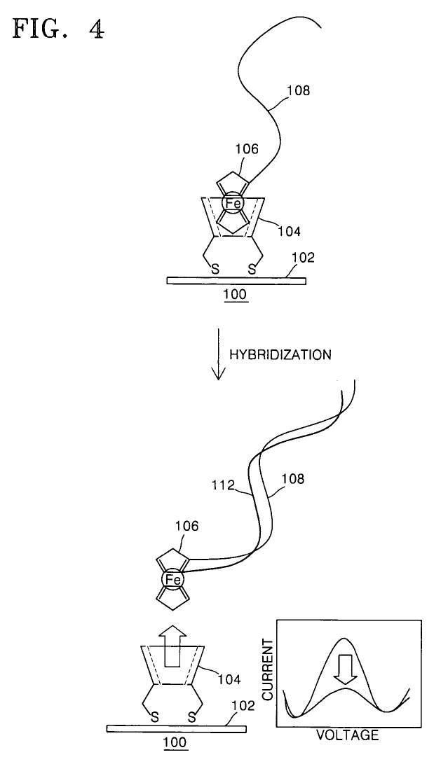

FIG. 4 is a diagram illustrating a method of detecting a biomolecule using a biomolecule detection sensor according to an embodiment of the present invention.

Referring to FIG. 4, when a target biomolecule 112 is present in a sample, a binding between the target biomolecule 112 and a probe biomolecule 108 occurs, and thus, a secondary material 106 is separated from a first material 104. This induces a change in current.

If no significant change in current is observed, it can be determined that any biomolecule or a target biomolecule is absent in a sample.

In the following Examples, cyclic voltammograms were measured and compared by providing a sample containing no DNAs, a sample containing DNAs perfectly matching with probe DNAs, and a sample containing DNAs perfectly mismatching with probe DNAs, to a biomolecule detection sensor of the present invention, and the results are shown in FIGS. 6 through 12.

The results of FIGS. 6 through 12 show that a biomolecule detection sensor according to the present invention can easily detect a target biomolecule with high accuracy within a short time.

Hereinafter, the present invention will be described more specifically with reference to the following Examples. The following Examples are for illustrative purposes and are not intended to limit the scope of the present invention.

### EXAMPLE 1

Manufacturing of Biomolecule Detection Sensors According to the Present Invention

Biomolecule detection sensors according to the present invention were manufactured using a method illustrated in FIG. 2.

First, substrates made of gold (Au) were washed with deionized water for 5 minutes, methanol for 5 minutes, acetone for 3 minutes, and then deionized water for 5 minutes, to completely remove impurities from the substrates.

Next, hydroxyl groups at lower ends of β-cyclodextrins were substituted by thiol groups. The thiol-modified β-cyclodextrins were placed on the substrates and incubated in a wet chamber overnight so that the thiol-modified β-cyclodextrins were self-assembled on the substrates.

Next, thiol-modified straight alkane chains C₆ (1% mercaptohexanol) where one ends of straight alkane chains C₆ were substituted by thiol groups were provided on surfaces of the substrates to block exposed Au surfaces of the substrates. Then, the substrates were washed twice with deionized water for 10 minutes and once with methanol and dried so that the exposed surfaces of the substrates were capped.

Next, probe biomolecules (SEQ ID NO: 1) were immobilized to ferrocenes via linkers, —CO₂NH(CH₂)₆—, to prepare ferrocene-CO₂NH(CH₂)₆-probe biomolecules.

Next, the ferrocene-CO₂NH(CH₂)₆-probe biomolecules were provided on the β-cyclodextrin-immobilized substrates in water so that the β-cyclodextrins were selectively bound to ferrocenes. This completes biomolecule detection sensors according to the present invention.

### EXAMPLE 2

Measurement of Surface Reflectance in Individual Steps of Biomolecule Detection Sensor Manufacturing Method According to the Present Invention

Surface reflectance in the individual steps of the biomolecule detection sensor manufacturing method according to Example 1 was measured.

The measurement of surface reflectance was performed using Surface-Plasmon Resonance (SPR) equipment.

FIG. 3A is a graph illustrating a change in surface reflectance during the immobilization of the β-cyclodextrins and the surface capping of the substrates in the biomolecule detection sensor manufacturing method according to Example 1.

In FIGS. 3A through 3C, Rₒ is an initial reflectance, and R is a final reflectance.

Referring to FIG. 3A, there was a significant difference in relative reflectance before and after the immobilization of the β-cyclodextrins, whereas there was no significant difference in relative reflectance before and after the capping.

FIG. 3B is a graph illustrating a change in surface reflectance with respect to time and incident angle during the selective binding of the ferrocenes with the β-cyclodextrins in the biomolecule detection sensor manufacturing method according to Example 1, and FIG. 3C is a graph illustrating a change in surface reflectance with time during the selective binding of the ferrocenes with the β-cyclodextrins in the biomolecule detection sensor manufacturing method according to Example 1.

Referring to FIGS. 3A and 3B, the selective binding of the ferrocenes with the β-cyclodextrins was saturated about 6 minutes after providing the ferrocenes.

### EXAMPLE 3

Evaluation of Effect of Hybridization Buffer on Selective Binding

An effect of a hybridization buffer commonly used in DNA hybridization on a selective binding between a first material and a secondary material in a biomolecule detection sensor according to the present invention was evaluated.

In detail, during the selective binding between the β-cyclodextrins and the ferrocenes of Example 1, 0.2M Na₂SO₄, which was an electrolyte solution commonly used in binding between β-cyclodextrin and ferrocene, was used instead of water. At this time, the ferrocenes were used in a concentration of 50 μM.

Also, during the selective binding between the β-cyclodextrins and the ferrocenes of Example 1, a hybridization buffer, 1× SSPET(0.9 M NaCl, 60 mM NaH₂PO₄, 6 mM EDTA, pH 7.4, 0.005% Triton X-100 (Sigma)) was used instead of water. At this time, the ferrocenes were used in a concentration of 50 μM.

In the above two experiments, the gold substrates of the biomolecule detection sensors were used as operating electrodes, and Ag/AgCl electrodes as reference electrodes. A predetermined voltage was applied to the operating electrodes, and a change in redox current was measured to obtain cyclic voltammograms. The results are shown in FIG. 5.

FIG. 5 is a graph illustrating redox peaks in the hybridization buffer and the electrolyte solution of Example 3.

Referring to FIG. 5, redox peaks in the 0.2M Na₂SO₄ electrolyte solution and in the 1× SSPET hybridization buffer, used instead of water, exhibited nearly identical shapes. This shows that a 1× SSPET hybridization buffer does not adversely affect a selective binding between β-cyclodextrin and ferrocene, and thus, a cyclic voltammogram can be obtained even without using an electrolyte solution.

### EXAMPLE 4

Detection of Target Biomolecules Using Biomolecule Detection Sensors According to the Present Invention

It was determined whether the biomolecule detection sensors manufactured in Example 1 could effectively detect target biomolecules.

For this, cyclic voltammograms with time when a hybridization buffer containing no DNA, a hybridization buffer containing target DNAs (CAA GAC AAG AGA ACA: SEQ ID NO: 2) perfectly matching with probe DNAs (Cp₂FeCO₂NH(CH₂)₆-TGT TCT CTT GTC TTG: SEQ ID NO: 1), and a hybridization buffer containing DNAs (TTT TTT TTT TTT TTT: SEQ ID NO: 3) perfectly mismatching with the probe DNAs were respectively provided to the biomolecule detection sensors manufactured in Example 1 were obtained. The cyclic voltammograms were obtained using the method described in Example 3.

A 1× SSPET solution was used as the hybridization buffer, the perfectly matched target DNAs and the perfectly mismatched DNAs were each used in a concentration of 100 nM, and the hybridization was performed at a temperature of 20° C.

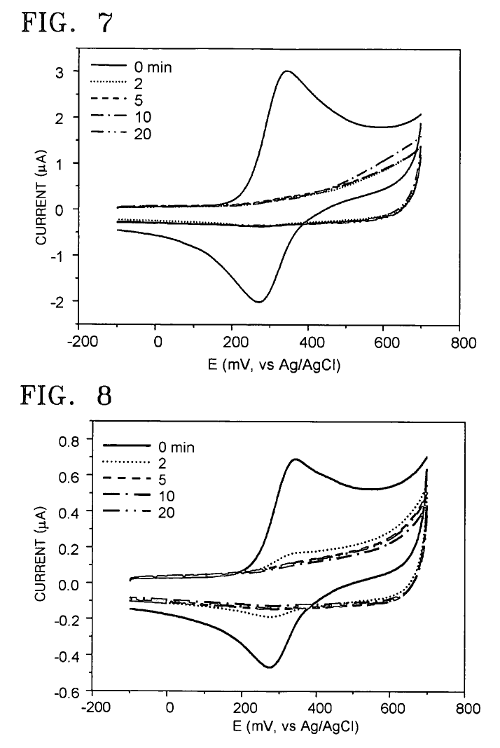

FIG. 6 is a cyclic voltammogram with time when the hybridization buffer containing no DNA is provided to the biomolecule detection sensors manufactured in Example 1, FIG. 7 is a cyclic voltammogram with time when the hybridization buffer containing the perfectly matched target DNAs is provided to the biomolecule detection sensors manufactured in Example 2, and FIG. 8 is a cyclic voltammogram with time when the hybridization buffer containing the perfectly mismatched DNAs is provided to the biomolecule detection sensors manufactured in Example 1.

Referring to FIG. 6, a cyclic voltammogram for the gold substrates having no ferrocene for immobilization of probe biomolecules is provided as a control (β-CD Au electrode without Fc-ssDNA).

At about 10 minutes after the hybridization buffer containing no DNA was provided to the biomolecule detection sensors of Example 1, the cyclic voltammogram for the hybridization buffer became similar to that of the control.

Referring to FIG. 7, at about 2 minutes after the hybridization buffer containing the perfectly matched target DNAs was provided to the biomolecule detection sensors of Example 1, the cyclic voltammogram for the hybridization buffer became similar to that of the control.

Referring to FIG. 8, at about 5 minutes after the hybridization buffer containing the perfectly mismatched DNAs was provided to the biomolecule detection sensors of Example 1, the cyclic voltammogram for the hybridization buffer became similar to that of the control.

These results show that ferrocene immobilized with a probe biomolecule is completely released from β-cyclodextrin by diffusion at a predetermined time (10 minutes in FIG. 6, 2 minutes in FIG. 7, and 5 minutes in FIG. 8) after a sample is provided to a biomolecule detection sensor.

Thus, the presence or concentration of a target biomolecule can be effectively determined based on a cyclic voltammogram obtained at about 2 minutes after a sample is provided to a biomolecule detection sensor according to the present invention.

### EXAMPLE 5

Measurement of ΔE in Biomolecule Detection Sensors According to the Present Invention

A difference (ΔE) between oxidation peak and reduction peak was measured using the three cyclic voltammograms obtained in Example 4.

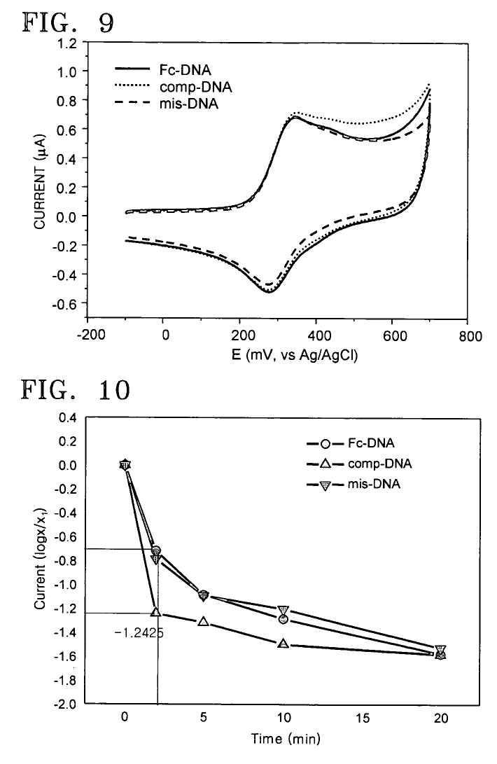

FIG. 9 illustrates a comparison of the cyclic voltammograms of FIGS. 6 through 8 (Fc-DNA: FIG. 6, comp-DNA: FIG. 7, mis-DNA: FIG. 8).

ΔE for the cyclic voltammograms of FIGS. 6 through 9 was measured and the results are presented in Table 1 below.

TABLE 1

| Sample | Ea (mV) | Ec (mV) | ΔE (mV) |
|---|---|---|---|
| Fc-DNA | 343 | 277 | 66 |
| comp-DNA | 349 | 275 | 74 |
| mis-DNA | 343 | 277 | 66 |

In Table 1, Ea is an oxidation peak, Ec is a reduction peak, and ΔE is a difference between Ea and Ec.

The results of Table 1 show that a hybridization buffer containing a perfectly matched target DNA affects the oxidation and reduction of ferrocene in a biomolecule detection sensor, unlike a hybridization buffer containing no DNA and a hybridization buffer containing a perfectly mismatched DNA.

### EXAMPLE 6

Measurement of Change in Current in Biomolecule Detection Sensors According to the Present Invention

A change in current with time for the three cyclic voltammograms obtained in Example 4 was measured. For this, an applied voltage was 500 mV/s (from −100 to 700 mV).

FIG. 10 is a graph illustrating a change in current with time for the cyclic votammograms of FIGS. 6 through 8 (Fc-DNA: FIG. 6, comp-DNA: FIG. 7, mis-DNA: FIG. 8).

In FIG. 10, a change in current is represented by log(x/x₁) where x₁ is an initial current measurement and x is a current measurement after a predetermined time, i.e., 2 minutes, 5 minutes, 10 minutes, and 20 minutes.

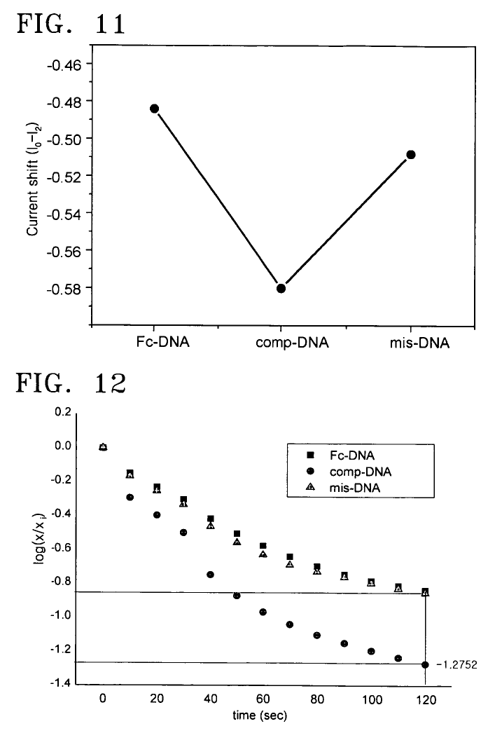

FIG. 11 is a graph illustrating a current shift for 2 minutes for the cyclic votammograms of FIGS. 6 through 8 (Fc-DNA: FIG. 6, comp-DNA: FIG. 7, mis-DNA: FIG. 8).

In FIG. 11, a current shift is represented by I₀-I₂ where I₀ is an initial current measurement, and I₂ is a current measurement after 2 minutes.

Referring to FIGS. 10 and 11, a change in current when using the hybridization buffer containing the perfectly matched DNAs was significantly different from that when using the hybridization buffer containing no DNA and the hybridization buffer containing the perfectly mismatched DNAs.

FIG. 12 is a graph illustrating a current measured every 10 seconds for 2 minutes for the cyclic votammograms of FIGS. 6 through 8 (Fc-DNA: FIG. 6, comp-DNA: FIG. 7, mis-DNA: FIG. 8).

Referring to FIG. 12, the cyclic voltammogram measurements obtained every 10 seconds for 2 minutes were similar to those of FIG. 10, and a signal change greater than an error range was observed.

In comparison between the cyclic voltammogram measurements obtained at 2 minutes after providing the hybridization buffer (FIG. 10) and the cyclic voltammogram measurements obtained every 10 seconds for 2 minutes after providing the hybridization buffer (FIG. 12), with respect to Fc-DNA, a difference between a change in the cyclic voltammogram measurements of FIG. 10 and a change in the cyclic voltammogram measurements of FIG. 12 was 0.1. On the other hand, with respect to comp-DNA, a change in the cyclic voltammogram measurements of FIG. 10 was −1.2425, which was nearly identical to a change (−1.2752) in the cyclic voltammographic measurements of FIG. 12. It can be seen from the results that diffusion with time is more influential than the number of cyclic voltammogram measurements.

These results show that a biomolecule detection sensor according to the present invention can easily detect a target DNA with accuracy within a short time (e.g., within 2 minutes).

While the present invention has been particularly shown and described with reference to exemplary embodiments thereof, it will be understood by those of ordinary skill in the art that various changes in form and details may be made therein without departing from the spirit and scope of the present invention as defined by the following claims. Thus, the embodiments must be employed for descriptive purposes, not for restrictive purposes. The scope of the present invention is defined by the following claims, not by the above descriptions. Thus, it must be understood that the present invention covers equivalents, alternatives, etc. falling within the scope of the present invention.

As described above, according to the present invention, a target biomolecule can be easily detected using an electrochemical reaction with high accuracy within a short time. Furthermore, there is no need to elaborately design probes, and thus it is easy to manufacture a detection sensor. In addition, label-free detection is possible, which simplifies a detection process, and there is no need to use expensive auxiliary equipment.

What is claimed is:

1. A sensor for detecting a biomolecule, the sensor comprising:
a substrate;
a first material, immobilized on the substrate, having a cavity structure;
a secondary material capable of selectively binding to the cavity structure of the first material and having electrochemical activity; and
a probe biomolecule immobilized to the secondary material.

2. The sensor of claim 1, wherein the substrate is selected from the group consisting of silicone wafer, glass, quartz, metal, and plastic.

3. The sensor of claim 1, wherein gold is coated on a surface of the substrate.

4. The sensor of claim 1, wherein the first material is cyclodextrin or calixarene, and the secondary material is metallocene, alkylammonium, or a compound containing an adamantly group.

5. The sensor of claim 1, wherein the first material is β-cyclodextrin and the secondary material is ferrocene.

6. The sensor of claim 1, wherein the first material is immobilized on the substrate using a self-assembly process.

7. The sensor of claim 1, wherein the biomolecule is a nucleic acid or a protein.

8. The sensor of claim 7, wherein the nucleic acid is selected from the group consisting of DNA, RNA, Peptide Nucleic Acid (PNA), Locked Nucleic Acid (LNA), and a hybrid thereof.

9. A method of manufacturing a biomolecule detection sensor, the method comprising:
immobilizing a first material having a cavity structure on a substrate;
immobilizing a probe biomolecule to a secondary material capable of selectively binding to the cavity structure of the first material and having electrochemical activity; and
providing the secondary material to the first material so that the secondary material selectively binds to the first material.

10. The method of claim 9, further comprising providing a capping material to the substrate so that a first material-free surface of the substrate is capped by the capping material, after the immobilization of the first material on the substrate.

11. The method of claim 9, wherein the first material is β-cyclodextrin and the secondary material is ferrocene.

12. The method of claim 9, wherein the immobilization of the first material on the substrate is performed using a self-assembly process.

13. A method of detecting a biomolecule, the method comprising:
providing a sample suspected of containing a target biomolecule to a biomolecule detection sensor comprising a substrate; a first material, immobilized on the substrate, having a cavity structure; a secondary material capable of selectively binding to the cavity structure of the first material and having electrochemical activity; and a probe biomolecule immobilized to the secondary material; and
measuring a change in current before and after providing the sample.

14. The method of claim 13, wherein the measuring of the change in current is performed using cyclic voltammetry.

15. The method of claim 13, wherein when the target biomolecule is present in the sample, the target biomolecule binds to the probe biomolecule, the secondary material is separated from the first material, and a current is changed.
